(19) United States
(12) Patent Application Publication
(10) Pub. No.: US 2009/0130200 A1
(43) Pub. Date: May 21, 2009

(54) GRANULES FOR CONTROLLED RELEASE OF TAMSULOSIN

(75) Inventors: Eszter Julianna Biro, Frick (CH); Waltraud Bueb, Aarburg (CH)

Correspondence Address: BRINKS HOFER GILSON & LIONE, P.O. BOX 10395, CHICAGO, IL, 60610, US

(73) Assignee: SIEGFRIED GENERICS INTERNATIONAL AG, Zofingen (CH)

(21) Appl. No.: 11/632,327

(22) PCT Filed: Jul. 7, 2005
(86) PCT No.: PCT/EP2005/007349

§ 371 Date: Jan. 7, 2009

(30) Foreign Application Priority Data
Jul. 14, 2004 (EP) 04016523.5

(51) Int. Cl. A61K 9/48; A61K 31/18

(52) U.S. Cl. 424/456; 514/603

(57) ABSTRACT

Granules for controlled release of Tamsulosin, the granules including Tamsulosin and a carrier matrix. The carrier matrix includes a) 2 to 25% by weight of an alginate and b) 30 to 70% by weight of a macromolecular substance selected from the group consisting of: methacrylic acid/ethyl acrylate 1:1 copolymer, methacrylic acid/methyl methacrylate 1:1 or 1:2 copolymers, aminoalkyl methacrylate copolymer, vinyl acetate/crotonic acid copolymer, polyvinyl acetate phthalate, ethylene-vinyl acetate, cellulose acetate phthalate, hydroxypropylmethylcellulose, sodium carboxymethylcellulose, carrageenan, crosslinked chitosan, polyethylene-vinyl acetate, poly-L-lactic acid, xanthan gum, polyvinyl acetate and mixtures thereof. The carrier matrix further includes 10 to 50% by weight of a hydrophobic substance selected from the group consisting of: glycerol behenate, glyceryl monostearate, wax, mono-, di- and trisubstituted glycerides, calcium stearate and mixtures thereof.

WO 03/009831 describes a rapidly disintegrating tablet from granules with delayed release of active ingredient.

EP 1043031 describes a gel formulation comprising ionic pharmaceutical substances such as, for example, Tamsulosin and oppositely charged excipients.

WO 01/78725 describes a process for producing spherical pellets. This entails mixing solvent, active ingredient and at least one carrier, the solvent not being sprayed on. The mixture is stirred, the solvent is removed and the pellets obtained in this way are dried.

DE 202 19 293 U1 discloses a pellet having a core and a coating layer, the core comprising Tamsulosin, microcrystalline cellulose, an acryl polymer and water. The coating layer comprises a acid resistant acryl polymer.

WO 2004/056354 discloses a controlled release pharmaceutical composition of Tamsulosin. Said controlled release pharmaceutical composition is a pellet comprising a core and an enteric coating over said core.

WO 2004/040064 discloses a pellet prepared by dissolving Tamsulosin and hydrophilic polymer to a mixture, combining the mixture with an inert diluent to form a combination having suitable ductility and extruding the combination to a filament followed by molding the filament to a pellet.

U.S. Pat. No. 4,772,475 discloses a pharmaceutical controlled release individual unit or multiple unit formulation comprising a granulation product obtained by adding a release controlling agent to a mixture of a physiologically active substance and units-forming substances and granulation and resultant mixture. The granulation product is substantially not disintegrated but gradually releasing the physiologically active substance in the gastrointestinal tract.

WO 96/26717 and WO 99/39698 disclose sustained release formulation comprising alginate, an enteric polymer, a pH independent gelling polymer as well as a drug. Both formulations have a high drug concentration.

It is an object of the present invention to provide an alternative administration form to pellets comprising Tamsulosin and a process for the production thereof, the administration form being intended to have uniform distribution of active ingredient and a controlled release profile.

The object is achieved by granules as claimed in claim 1 and the process as claimed in claims 12. The dependent claims relate to further preferred embodiments.

The granules comprise Tamsulosin and a carrier matrix. The carrier matrix comprises 2 to 25% by weight of an alginate, 30 to 70% by weight of a macromolecular substance and 10 to 50% by weight of a hydrophobic substance. The combination of these components as carrier matrix makes it possible for the release of Tamsulosin to be time- and pH-dependent and thus for the release profile in vivo to be optimal. The granules of the invention have a homogeneous distribution of active ingredient through the choice of the carrier matrix, even if the active ingredient is present in only small amounts.

The Tamsulosin present in the granules of the invention is normally the R enantiomer of Tamsulosin, but the S enantiomer or a mixture of the two in various ratios would also be possible. Tamsulosin may be present as free base or as salt in the granules. Possible salts are Tamsulosin hydrochloride, Tamsulosin besylate, Tamsulosin acetate, Tamsulosin maleate, Tamsulosin tartrate and Tamsulosin citrate. The hydrochloride salt is preferably present in the granules of the invention.

The amount of Tamsulosin present in the granules of the invention is relatively low, generally lower than 10 mg, preferably between 0.1 and 1.2 mg and particularly preferably between 0.2 and 0.8 mg based on the active ingredient employed. In a preferred embodiment, the granules of the invention comprise 0.4 mg of Tamsulosin hydrochloride.

The alginate present in the carrier matrix of the granules of the invention is selected from the group of sodium alginate and propylene glycol alginate. Sodium alginate is a mixture of polyuronic acids consisting of D-mannuronic acid and D-guluronic acid. The ratio of D-mannuronic acid and D-guluronic acid may vary and influences the gel property of the carrier matrix and the porosity of the gel. It is preferred for the relative proportion of D-mannuronic acid to be from 58 to 62% by weight and the relative proportion of D-guluronic acid to be from 38 to 42% by weight. In the stomach, the hydrated sodium alginate is converted into a porous, insoluble layer. In the intestine, that is at a pH above 5.5, this porous, insoluble layer becomes a soluble viscous layer. If the granules of the invention comprise propylene glycol alginate, they have exceptionally good stability under acidic conditions. At a higher pH, that is in the intestine, the propylene glycol groups are eliminated by hydrolysis.

The macromolecular substance is selected from the group of methacrylic acid/ethyl acrylate 1:1 copolymer, methacrylic acid/methyl methacrylate 1:1 or 1:2 copolymers, aminoalkyl methacrylate copolymers, vinyl acetate/crotonic acid copolymers, polyvinyl acetate phthalate, ethylene-vinyl acetate, cellulose acetate phthalate, hydroxypropylmethylcellulose, sodium carboxymethylcellulose, carrageenan, crosslinked chitosan, polyethylene-vinyl acetate, poly-L-lactic acid, xanthan gum and polyvinyl acetate or mixtures thereof. The granules of the invention preferably comprise methacrylic acid/ethyl acrylate copolymer because this confers on the granules a good mechanical strength. Methacrylic acid/ethyl acrylate copolymer is known under the proprietory name of Eudragit L-100-55. Preference is likewise given to polyvinyl acetate, which leads to a very coherent carrier matrix, and polyvinyl acetate phthalate, which leads to a very good pH-dependent release of Tamsulosin.

The hydrophobic substance which is likewise present in the granules of the invention is selected from the group of glycerol behenate, glyceryl monostearate, wax, mono-, di- and trisubstituted glycerides and calcium stearate or mixtures thereof. Glycerol behenate is preferred in this connection. Glyceryl behenate is a mixture of glycerides of fatty acids, mainly of behenic acid. There are various glycerol behenates such as, for example, 1,2,3-propanetriyl ester and Compritol® 888 ato, which consists of mono-, di- and triglycerides, with the proportion of diglycerides being predominant. Glycerol behenate serves as release-slowing agents so that the release of Tamsulosin from the granules of the invention takes place in a delayed manner.

The granules of the invention are preferably contained in a hard gelatin capsule which dissolves in the gastric region. The active ingredient is released in small amounts in the gastric region, but especially in the intestinal region.

In a preferred embodiment, the granules of the invention additionally comprise a binder selected from the group of maltodextrin, polyvinylpyrrolidone, pregelatinized starch and sodium starch glycolate. Particular preference is given to maltodextrin, a maltooligosaccharide which is produced by partial hydrolysis of starch. It is a natural binder, is soluble in cold water and has excellent wettability. The binder preferably has a concentration of from 2 to 25% by weight, particularly preferably from 12 to 18% by weight.

In a further embodiment, the granules of the invention additionally comprise a surface-active agent selected from the group of sodium lauryl sulfate, poloxamer, polyoxyethylene stearate and polyoxyethylene castor oil derivatives such as, for example, polyoxyl 40 hydrogenated castor oil. Since Tamsulosin hydrochloride is an active ingredient which is only slightly soluble in water, a surface-active agent can be added to improve the solubility. Sodium lauryl sulfate is particularly preferred. This anionic, surface-active agent increases the solubility of Tamsulosin significantly and can accompany the release of Tamsulosin in the intestine through the more or less damaged structure of the granules. The surface-active agent preferably has a concentration of from 0.5 to 10% by weight, particularly preferably from 1 to 4% by weight.

In a further embodiment, the granules of the invention comprise a plasticizer selected from the group of Makrogol 6000, Polysorbate 80, triethyl citrate, acetyl triethyl citrate, tributyl citrate, propylene glycol, diethyl phthalate, triacetin, acetyl tributyl citrate and dibutyl sebacate. The presence of a plasticizer reduces the film forming temperature and the glass transition temperature of the methacrylic acid/ethyl acrylate copolymer and of the polyvinyl acetate. Makrogol 600 and Polysorbate 80 are particularly preferred in this connection. Makrogol is a condensation polymer of ethylene oxide and water. The number 6000 stands for the average molecular weight. Polysorbate 80 results in the esterification of sorbitol and of its anhydrides with oleic acid and subsequent reaction with ethylene oxide. Each mol of sorbitol or its anhydride has about 20 mol of ethylene oxide. Polysorbate 80 additionally has a dispersion-stabilizing effect. The plasticizer preferably has a concentration of from 0.5 to 5% by weight, particularly preferably from 0.5 to 2% by weight.

Further excipients such as a pH-adjusting agent, an antifoam and/or a glidant can be added to the granules of the invention.

An example of a possible pH-adjusting agent is sodium hydroxide. The pH of the granulating liquid is in this case preferably adjusted to a pH of approximately 5 by adding small amounts of sodium hydroxide or another base known to the skilled worker. Some of the carboxylic acid groups of the macromolecular substance are neutralized thereby, and the redispersibility thereof is improved thereby.

Addition of an antifoam simplifies the granulation process. An example thereof is simethicone, which reduces the surface tension. The antifoam is preferably employed in amounts of from 1 to 100 ppm.

The flowability of the granules can be increased by the presence of a glidant such as, for example, colloidal anhydrous silica. The glidant preferably has a concentration of from 0.1 to 2% by weight.

The granules of the invention may comprise liquids such as water and solvents, and mixtures thereof. Possible liquids are ethanol, ethanol/water, isopropyl alcohol, isopropyl alcohol/water, n-butanol, acetone, acetone/ water, acetone/isopropyl alcohol. Suitable mixing ratios are known to the skilled worker.

In a particularly preferred embodiment, the granules of the invention comprise from 0.15 to 0.35% by weight of Tamsulosin, 6 to 8% by weight of sodium alginate, 55 to 65% by weight of methacrylic acid/ethyl acrylate 1:1 copolymer, 12 to 18% by weight of glycerol behenate, 12 to 18% by weight of maltodextrin and 0 to 10% by weight of excipients. It is presumed that during the release of Tamsulosin these granules are penetrated and hydrated by liquid. This results in partial swelling of the alginate, after which the dissolved Tamsulosin is able to diffuse slowly through the resulting gel layer, and the alginate layer is degraded in the intestinal tract. The hydrophobic glyceryl behenate slows the penetration of liquid into the gel layer. Methacrylic acid/ethyl acrylate copolymer forms in part a polymer film over and between the granules, which slows the release of Tamsulosin in an acidic environment. The presence of the methacrylic acid/ethyl acrylate copolymer confers good mechanical strength on the granules of the invention and controls the diffusion of the Tamsulosin during its release. The release profile of these granules is excellent.

The granules of the invention are produced by using a standard granulation technique using a high speed mixer (high shear mixer). This entails production in two steps of a powder mixture and of a granulating liquid. The powder mixture comprises the alginate, part of the macromolecular substance, the hydrophobic substance and optionally a binder. These components are mixed until a homogeneous powder mixture is obtained.

The granulating liquid is produced by adding the remaining part of the macromolecular substance and the Tamsulosin to the water or the solvent. The liquid is stirred until a homogeneous solution is obtained. In a preferred embodiment, the water or the solvent is heated and, in a first step, a plasticizer is added and mixed. The solution containing the plasticizer is then cooled. The flexibility of the granulating liquid and the coating efficiency is increased through the presence of the plasticizer. At least one surface-active agent, Tamsulosin and optionally an antifoam and/or a glidant are put in a separate container and likewise mixed until a homogeneous solution is produced. The remaining part of the macromolecular substance is added to this solution and stirred further. Finally, the solution containing the plasticizer is slowly added to this mixture and optionally a pH-adjusting agent is also added so that a homogeneous granulating liquid is produced.

The homogeneous granulating liquid is rapidly added with vigorous stirring to the homogeneous powder mixture. The vigorous stirring is essential in order to be able to ensure that the active ingredient is well dispersed and the particle size is approximately homogeneous. This is important because of the low concentration of the Tamsulosin active ingredient, because it can be ensured in this way that each capsule has the same active ingredient concentration. Alternatively, the granulating liquid can be sprayed on. If the small particles are sprayed with a granulating liquid of medium or low viscosity, the Tamsulosin is optimally dispersed in the granules, so that active ingredient concentration is uniform.

The granules are then dried. This can take place for example in a drying oven with or without vacuum, in a circulating air dryer, in a one-pot granulator or in a fluidized bed system. The dry granules are then processed with a hammer mill or screened using an oscillating or rotating sieve in order thus to obtain a fine particle distribution. Particularly good results and a narrow particle distribution were obtained with a hammer mill.

The figures mentioned in the following examples show:

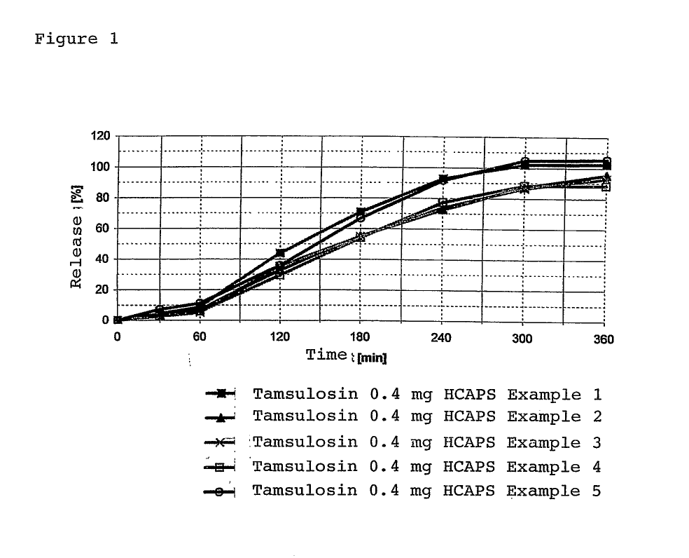

FIG. 1: a plot of the release of Tamsulosin from the granules according to examples 1 to 5,

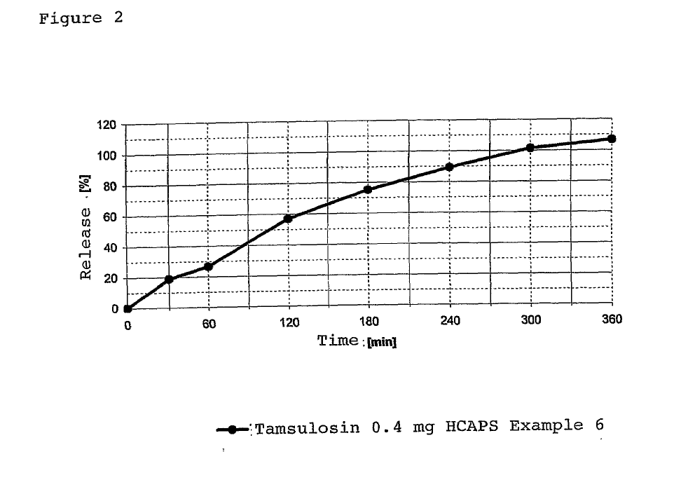

FIG. 2: a plot of the release of Tamsulosin from the granules according to example 6,

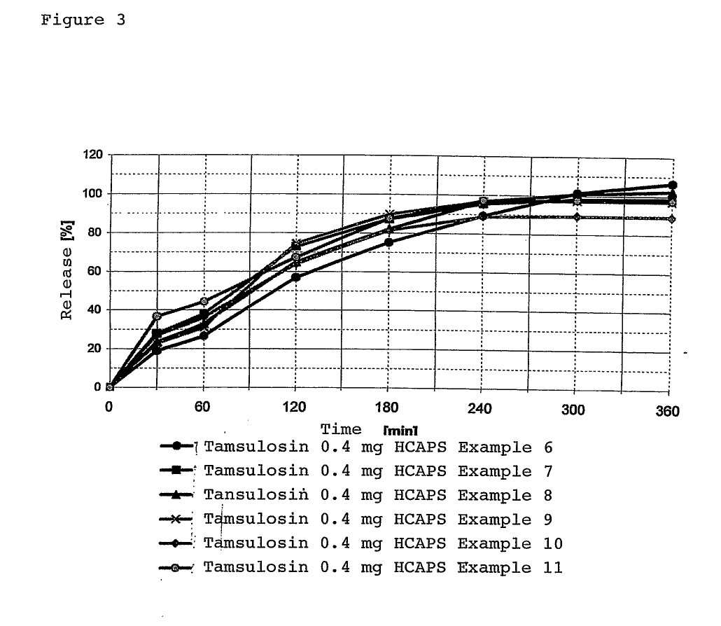

FIG. 3: a plot of the release of Tamsulosin from the granules according to examples 6 to 11,

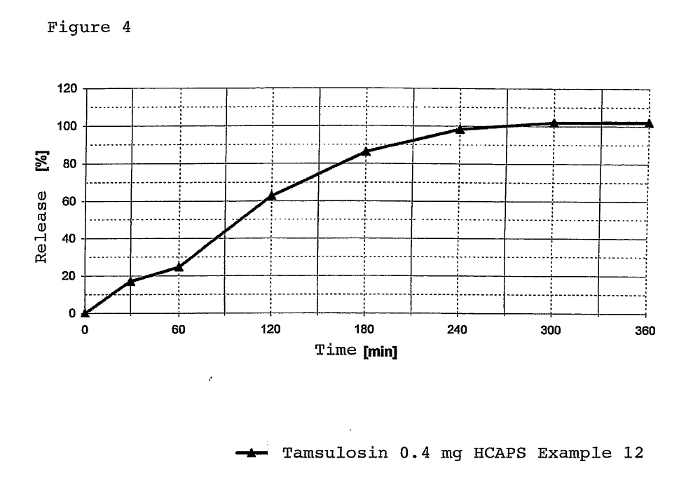

FIG. 4: a plot of the release of Tamsulosin from the granules according to example 12,

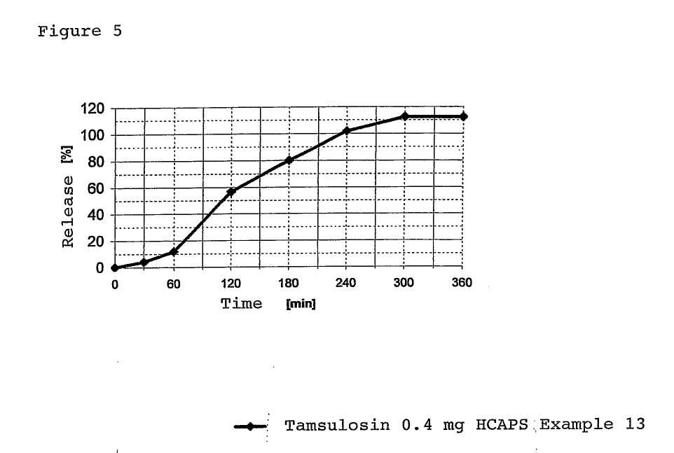

FIG. 5: a plot of the release of Tamsulosin from the granules according to example 13.

### EXAMPLES

### Investigation of Tamsulosin Release

### Test Method:

The release test was carried out by the rotating basket method at a speed of rotation of 50 rpm. In the method, the conditions in the gastrointestinal tract were simulated by changing the media after a certain time in order to simulate the pH gradient. 450 ml of medium 1 were left for one hour, and then 150 ml of medium 2 were added.

| | |
|---|---|
| Medium 1: | 0.1 N HCl |
| Medium 2: | 0.05 M USP buffer of pH 6.8 |
| | 6.8 g of KH₂PO₄ |
| | 22.4 ml of 0.2 M NaOH |
| | make up to 200 ml with water |

### Apparatus and Analysis Conditions:

Apparatus of Ph. Eur. (current edition (2004), rotating basket apparatus)

Method: The capsules are first put into 450 ml of 0.1 N HCl for 1 hour. Then 150 ml of the sodium phosphate buffer solution, which had been equilibrated at 37° C., are added in order thus to obtain a buffer solution with a pH of 6.8. Before starting the release test, 450 ml of 0.1 N HCl and 150 ml of sodium phosphate buffer are mixed, and the pH of this solution is measured. If necessary, the pH of the sodium phosphate buffer was adjusted in order to obtain a pH of 6.8±0.05 for the solution.

| | |
|---|---|
| Volume: | 450 ml (first hour) → 600 ml |
| Medium 1: | 100 ml of 1 N hydrochloric acid were diluted to |
| | 1000 ml with water |
| Medium 2: | 450 ml of HCl 0.1 N + 150 ml of sodium phosphate |
| | buffer |
| Sodium phosphate | 91 g of Na₃PO₄ * 12 H₂O are dissolved in water |
| buffer: | and diluted to 1000 ml of water. |
| Stirring speed: | 50 rpm |
| Temperature: | 37° C. ± 0.5° C. |
| Sampling time: | After 0.5, 1, 2, 3, 4, 5 and 6 hours, about 2 ml of |
| | each test solution were removed, and the amount |
| | of dissolved Tamsulosin was determined by the |
| | HPLC method indicated below. |

### HPLC Method to Determine Released Tamsulosin

### Preparation of the Reference Solution

| | |
|---|---|
| Reference stock | 20 to 24 mg of Tamsulosin HCl reference |
| solution: | substance were accurately weighed and put into a |
| | 100 ml graduated cylinder and diluted to the |
| | appropriate volume with 0.1 N hydrochloric acid. |
| | The graduated cylinder was put at room |
| | temperature into an ultrasonic bath until |
| | Tamsulosin HCl was completely dissolved. |
| Reference | 10.0 ml of the reference stock solution were put |
| solution 1: | into a 200 ml graduated cylinder and diluted to the |
| | volume with 0.1 N hydrochloric acid. 20.0 ml of the |
| | solution obtained in this way was put in a 250 ml |
| | graduated cylinder and diluted to the volume with |
| | 0.1 N hydrochloric acid. (Concentration of |
| | Tamsulosin HCl 0.88 μg/ml). |
| Reference | 10.0 ml of the reference stock solution was put in |
| solution 2: | a 200 ml graduated cylinder and diluted to the |
| | volume with medium 2. 15.0 ml of the solution |
| | obtained in this way was put in a 250 ml graduated |
| | cylinder and diluted to the volume with medium 2. |
| | (Concentration of Tamsulosin HCl 0.66 μg/ml). |

### Test of Suitability of the System for Release Analysis

The test of suitability for the system was carried out before the HPLC release test on the 0.4 mg Tamsulosin capsules.

Method: Reference solution 1 is injected 5 times, and the retention time is recorded in the form of a band. The calculated relative standard deviation (RSD) of the Tamsulosin bands is calculated. The end of the band (tailing) is determined for the band of the 5th injection.

| | |
|---|---|
| Specification: | RSD ≦ 2.0% |
| | end of band ≦ 1.5 |
| HPLC conditions: | |
| Column: | Nucleosil 100-5 C-18 AB, |
| | 125 × 3 mm, particle size |
| | 5 μm. |
| Solvent: | 780 ml of buffer, 220 ml of acetonitrile |
| Buffer: | H₃PO₄ is added to 0.05 M KH₂PO₄ |
| | until the pH is 3.0 ± 0.05. |
| Duration of analysis: | 5 minutes |
| Volume injected: | 50 μl |
| Column temperature: | 40° C. |
| Flow rate: | 1.0 ml/min |
| UV detection: | Signal: 225.4 nm, |
| | Reference: 550, 100 nm |

### Examples 1 to 5

The granules were produced by using a standard granulation technique with a high-shear granulator.

The powder mixture comprises a carrier matrix comprising sodium alginate, glycerol behenate and polyvinyl acetate phthalate and the binder maltodextrin.

The granulating liquid was an aqueous dispersion comprising polyvinyl acetate phthalate, dissolved Tamsulosin and the surface-active agent sodium lauryl sulfate and poloxamer, but other surface-agents acting as solubilizers could also have been employed.

During the wet kneading together, the granulating liquid formed granules by agglomeration of the powder particles. Drying of the granules was followed by screening thereof with an oscillating sieve. The dried granules were then packed into the hard gelatin capsules.

The release of Tamsulosin can be controlled within a wide range (see FIG. 1) by altering the amounts of the individual components in the carrier matrix. Hence, granules in which the amount of the individual components was varied were also investigated. Table 1 shows the composition of the granules.

TABLE 1
Examples 1-5

| | Example | | | | |
|---|---|---|---|---|---|
| | 1 | 2 | 3 | 4 | 5 |
| Composition | w/w % | w/w % | w/w % | w/w % | w/w % |
| Tamsulosin HCl* | 0.18 | 0.18 | 0.18 | 0.18 | 0.18 |
| Sodium lauryl sulfate* | 1.00 | 1.00 | 1.00 | 1.00 | 1.00 |
| Poloxamer 407* | 0.25 | 0.25 | 0.25 | 0.25 | 0.25 |
| Simethicone* | 0.01 | 0.01 | 0.04 | 0.04 | 0.04 |
| Maltodextrin | 10.00 | 10.00 | 10.00 | 12.00 | 28.53 |
| Sodium alginate | 10.00 | 20.00 | 7.50 | 12.00 | 10.00 |
| Glycerol dibehenate | 20.00 | 10.00 | 17.50 | 18.53 | 0.00 |
| Polyvinyl acetate | 58.56 | 58.56 | 63.53 | 56.00 | 60.00 |
| phthalate** | | | | | |

*in the granulating liquid
**partly in the granulating liquid

### Example 6

The same process was used as in Examples 1 to 5. In this case, the composition indicated in Table 2, column 2, was used. Polyvinyl acetate phthalate was replaced by methacrylic acid/ethyl acrylate copolymer (1:1). Addition of the plasticizer resulted in the dispersion of the methacrylic acid/ethyl acrylate copolymer (1:1) having the necessary flexibility. Macrogol 6000 was selected as plasticizer. The surface-active agent poloxamer 407 was replaced by polysorbate 80 because of its dispersion-stabilizing effect. This means that the homogeneity of the granulating liquid is improved. A sodium hydroxide solution was additionally used in order to adapt the pH of the polymer dispersion. The release profile is depicted in FIG. 2.

### Example 7 to 11

The same process as described in Example 6 was carried out using the granule compositions shown in Table 2 below. The sequence of addition of the ingredients and the composition of the granulating liquid were altered without any substantial alteration in the quantitative composition of the granules.

### Example 7

The amount of Eudragit was reduced to 15% in the granulating liquid (previously 20%)

### Example 8

The granulating liquid was produced without Eudragit, polysorbate and sodium hydroxide.

### Example 9

The sodium alginate powder was added after moistening of the powder mixture.

### Example 10

Less water in the granulating liquid

### Example 11

Granulating liquid was prepared without Eudragit, polysorbate, sodium hydroxide and macrogol.

TABLE 2

| | Example w/w % | | | | | |
|---|---|---|---|---|---|---|
| | 6 | 7 | 8 | 9 | 10 | 11 |
| Tamulosin HCl | *0.21 | *0.24 | *0.24 | *0.24 | *0.25 | *0.25 |
| Sodium alginate | 7.00 | 7.00 | 7.00 | 7.00 | 7.00 | 7.14 |
| Methacrylic | (9.67 + | #60.37 | #59.81 | #58.92 | 59.82 | 61.00 |
| acid/ethyl | 49.36) | | | | | |
| acrylate 1:1 | #59.03 | | | | | |
| copolymer | | | | | | |
| Glycerol | 15.00 | 15.00 | 15.00 | 15.00 | 15.00 | 15.29 |
| dibehenate | | | | | | |
| Maltodextrin | 15.00 | 15.00 | 15.00 | 15.00 | 15.00 | 15.29 |
| Sodium lauryl | *1.00 | *1.00 | *1.00 | *1.00 | *1.00 | *1.02 |
| sulfate | | | | | | |
| Macrogol 6000 | *1.94 | *0.97 | *1.94 | *2.00 | *1.92 | 0.00 |
| Polysorbate 80 | *0.76 | *0.39 | 0.00 | *0.80 | 0.00 | 0.00 |
| Sodium hydroxide | *0.05 | *0.02 | 0.00 | *0.03 | 0.00 | 0.00 |
| Simethicone | *0.01 | *0.01 | *0.01 | *0.01 | *0.01 | *0.01 |

*in the granulating liquid
**partly in the granulating liquid

As shown in FIG. 3, a smaller amount of Eudragit and/or water leads to fewer film regions in the granules, leading to faster release compared with the release profile of Example 6.

### Example 12

Tamsulosin-containing granules were produced by using the same production process as in Example 6 and using the composition shown in Table 3 (see FIG. 4).

The distribution of Tamsulosin was improved by changing the screening steps after drying. The oscillating sieve was replaced by a hammer mill. The flowability of the granules was increased by colloidal anhydrous silica being added as glidant and being mixed with the dried and screened granules.

TABLE 3

| Example w/w % | 12 |
|---|---|
| Tamsulosin HCl | *0.31 |
| Sodium alginate | 7.00 |
| Methacrylic acid/ethyl acrylate 1:1 copolymer | #58.94 |
| Glycerol dibehenate | 15.00 |
| Maltodextrin | 15.00 |
| Sodium lauryl sulfate | *1.15 |
| Macrogol 6000 | *1.69 |
| Polysorbate 80 | *0.65 |
| Sodium hydroxide | *0.05 |
| Simethicone | *0.01 |
| colloidal anhydrous silica | 0.20 |

*in the granulating liquid
#partly in the granulating liquid

### Example 13

Tamsulosin granules were produced by using the same process as in Example 6 and using the composition shown in Table 4, and additionally produced with a polymer film of methacrylic acid/ethyl acrylate 1:1 copolymer by a fluidized bed process. The release profile is depicted in FIG. 5.

TABLE 4

| Composition | w/w % |
|---|---|
| Matrix granules | |
| Granulating liquid | |
| Tamsulosin HCl | 0.17 |
| Methacrylic acid/ethyl acrylate 1:1 copolymer | 13.25 |
| Macrogol 6000 | 1.32 |
| Polysorbate 80 | 0.53 |
| Sodium lauryl sulfate | 0.63 |
| Sodium hydroxide | 0.07 |
| Simethicone | 0.01 |
| Powder mixture | |
| Microcrystalline cellulose | 20.00 |
| Calcium stearate | 4.00 |
| Pregelatinized acetylated distarch adipate | 38.02 |
| Film coating | |
| Methacrylic acid/ethyl acrylate 1:1 copolymer | 19.39 |
| Macrogol 6000 | 1.89 |
| Glycerol monostearate | 0.49 |
| Polysorbate 80 | 0.21 |
| Simethicone | 0.02 |
| Total | 100 |

### Example 14

The powder mixture comprises a carrier matrix comprising sodium alginate, glycerol dibehenate, Iota carrageenan, and polyvinyl acetate phthalate and the binder maltodextrin.

The granulating liquid was an aqueous dispersion comprising polyvinyl acetate phthalate, dissolved Tamsulosin and the surface-active agent sodium lauryl sulfate and triacetin.

During the wet kneading together, the granulating liquid formed granules by agglomeration of the powder particles. Drying of the granules was followed by processing the dry granules with a hammer mil in order to obtain a fine particle distribution. The dried granules were then packed into the hard gelatin capsules.

TABLE 5

| Composition: | % |
|---|---|
| Tamsulosin HCl* | 0.30 |
| Sodium Lauryl Sulphate* | 1.50 |
| Polyvinyl acetate phtalate** | 54.10 |
| Triacetin* | 0.75 |
| Sodium alginate | 2.15 |
| Iota carrageenan | 5.00 |
| Glycerol dibehenate | 14.40 |
| Maltodextrine | 21.30 |
| colloidal anhydrous silica | 0.50 |

*in the granulation liquid
**partly in the granulation liquid

### Example 15

The powder mixture comprises a carrier matrix comprising sodium alginate, glycerol dibehenate, xanthan gum, pregelatinized starch, and methacrylic acid-ethyl acrylate

copolymer 1:1.

The granulating liquid was an aqueous dispersion comprising methacrylic acid-ethyl acrylate copolymer 1:1, dissolved Tamsulosin and the surface-active agent sodium lauryl sulfate and macrogol 6000.

During the wet kneading together, the granulating liquid formed granules by agglomeration of the powder particles. Drying of the granules was followed by processing the dry granules with a hammer mil in order to obtain a fine particle distribution. The dried granules were then packed into the hard gelatin capsules.

TABLE 6:

| Composition: | % |
|---|---|
| Tamsulosin HCl* | 0.30 |
| Sodium lauryl sulphate* | 1.50 |
| Methacrylic acid - Ethyl acrylate | 62.00 |
| Copolymer(1:1)** | |
| Macrogol 6000* | 1.55 |
| Sodium alginate | 4.00 |
| Xanthan gum | 4.00 |
| Glycerol dibehenate | 10.00 |
| Pregelatinized starch | 16.15 |
| colloidal anhydrous silica | 0.50 |

*in the granulation liquid
**partly in the granulation liquid

### Example 16

The powder mixture comprises a carrier matrix comprising sodium alginate, cellulose acetate phthalate, calcium stearate, and polyvinylpyrrolidon.

The granulating liquid was an aqueous dispersion comprising cellulose acetate phthalate, dissolved Tamsulosin and the surface-active agent sodium lauryl sulfate and triacetin.

During the wet kneading together, the granulating liquid formed granules by agglomeration of the powder particles. Drying of the granules was followed by processing the dry granules with a hammer mil in order to obtain a fine particle distribution. The dried granules were then packed into the hard gelatin capsules.

TABLE 7

| Composition: | % |
|---|---|
| Tamsulosin HCl* | 0.30 |
| Sodium lauryl sulphate* | 1.50 |
| Cellulose acetate phtalate** | 66.40 |
| Triacetin* | 1.30 |
| Sodium alginate | 10.00 |
| Calcium stearate | 15.00 |
| Polyvinylpyrrolidon | 5.00 |
| colloidal anhydrous silica | 0.50 |

*in the granulation liquid
**partly in the granulation liquid

1-13. (canceled)

14. Granules for controlled release of Tamsulosin comprising: Tamsulosin and a carrier matrix, wherein the carrier matrix comprises:
a) 2 to 25% by weight of an alginate,
b) 30 to 70% weight of a macromolecular substance selected from the group consisting of: methacrylic acid/ethyl acrylate 1:1 copolymer, methacrylic acid/methyl methacrylate 1:1 copolymer, methacrylic acid/methyl methacrylate 1:2 copolymer, aminoalkyl methacrylate copolymer, vinyl acetate/crotonic acid copolymer, polyvinyl acetate phthalate, ethylene-vinyl acetate, cellulose acetate phthalate, hydroxypropylmethylcellulose, sodium carboxymethyl-cellulose, carrageenan, crosslinked chitosan, polyethylene-vinyl acetate, poly-L-lactic acid, xanthan gum, polyvinyl acetate and mixtures thereof, and
c) 10 to 50% by weight of a hydrophobic substance selected from the group consisting of: glycerol behenate, glyceryl monostearate, wax, mono-substituted glyceride, disubstituted glycerides, trisubstituted glycerides, calcium stearate, and mixtures thereof.

15. Granules as claimed in claim 14, wherein the macromolecular substance is methacrylic acid/ethyl acrylate 1:1 copolymer.

16. Granules as claimed in claim 14, wherein the hydrophobic substance is glycerol behenate.

17. Granules as claimed in claim 14, further comprising a binder selected from the group consisting of: maltodextrin, polyvinylpyrrolidone, pregelatinized starch, sodium starch glycolate, and mixtures thereof.

18. Granules as claimed in claim 14, further comprising a surface active agent selected from the group consisting of: sodium lauryl sulfate, poloxamer, polyoxyethylene stearate, polyoxyethylene castor oil derivatives, and mixtures thereof.

19. Granules as claimed in claim 14, further comprising a plasticizer selected from the group consisting of: condensation polymers of ethylene oxide and water, sorbitol oleic acid ester reacted with ethylene oxide, and anhydrides thereof, triethyl citrate, acetyl triethyl citrate, tributyl citrate, propylene glycol, diethyl phthalate, triacetin, acetyl tributyl citrate, dibutyl sebacate, and mixtures thereof.

20. Granules as claimed in claim 14, further comprising a pH-adjusting agent.

21. Granules as claimed in claim 14, further comprising an antifoam.

22. Granules as claimed in claim 14, further comprising a glidant.

23. Granules as claimed in claim 14, further comprising water.

24. Granules as claimed in claim 23, further comprising a solvent, wherein the solvent is selected from the group consisting of ethanol, isopropyl alcohol, and acetone.

25. Granules as claimed in claim 14, further comprising a mixture of water and a solvent.

26. Granules as claimed in claim 25, wherein the solvent is selected from the group consisting of ethanol, isopropyl alcohol and acetone.

27. Granules as claimed in claim 14, further comprising:
a) 0.15 to 0.35% by weight of Tamsulosin;
b) 6 to 8% by weight of sodium alginate;
c) 55 to 65% by weight of methacrylic acid/ethyl acrylate 1:1 copolymer;
d) 12 to 18% by weight of glycerol behenate;
e) 12 to 18% by weight of maltodextrin; and
f) 0 to 10% by weight of excipients.

28. A hard gelatin capsule comprising: granules for controlled release of Tamsulosin comprising: Tamsulosin and a carrier matrix, wherein the carrier matrix comprises:
a) 2 to 25% by weight of an alginate,
b) 30 to 70% by weight of a macromolecular substance selected from the group consisting of: methacrylic acid/ethyl acrylate 1:1 copolymer, methacrylic acid/methyl methacrylate 1:1 copolymer, methacrylic acid/methyl methacrylate 1:2 copolymer, aminoalkyl methacrylate copolymer, vinyl acetate/crotonic acid copolymer, polyvinyl acetate phthalate, ethylene-vinyl acetate, cellulose acetate phthalate, hydroxypropylmethylcellulose, sodium carboxymethylcellulose, carrageenan, crosslinked chitosan, polyethylene-vinyl acetate, poly-L-lactic acid, xanthan gum, polyvinyl acetate, and mixtures thereof, and
c) 10 to 50% by weight of a hydrophobic substance selected from the group consisting of: glycerol behenate, glyceryl monostearate, wax, monosubstituted glycerides, disubstituted glycerides, trisubstituted glycerides, calcium stearate, and mixtures thereof.

29. A process for producing granules comprising Tamsulosin, the process comprising:
a) mixing sodium alginate, a first part of a macromolecular substance, and a hydrophobic substance, resulting in a homogeneous powder mixture;
b) mixing water or a solvent, a second part of the macromolecular substance, and Tamsulosin, resulting in a homogeneous granulating liquid,
c) adding with vigorous stirring the homogeneous granulating liquid to the homogeneous powder mixture; and
d) drying the resulting granules.

30. The process as claimed in claim 29, wherein the homogeneous powder mixture is obtained by mixing the sodium alginate, the first part of the macromolecular substance, the hydrophobic substance, and a binder.

31. The process as claimed in claim 29, where the granulating liquid is obtained by performing the process comprising:
a) heating water or the solvent;
b) adding a plasticizer thereto and mixing,
c) cooling the solution obtained in this way and comprising the plasticizer;
d) mixing Tamsulosin and at least one surface-active agent, and adding to this mixture the second part of the macromolecular substance, mixing, and
e) adding the mixture obtained in the manner recited in process d) to the solution comprising the plasticizer obtained by the process recited in c).
